(19) United States
(12) Patent Application Publication
(10) Pub. No.: US 2006/0088942 A1
(43) Pub. Date: Apr. 27, 2006

(54) Measuring nitric oxide in blood gases and treatments based thereon

(76) Inventors: Jonathan S Stamler, Chapel Hill, NC (US); Timothy J McMahon, Durham, NC (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/516,250

(22) PCT Filed: May 27, 2003
(86) PCT No.: PCT/US03/13409

§ 371 Date: Aug. 25, 2005

Related U.S. Application Data
(60) Provisional application No. 60/383,595, filed on May 29, 2002.

(51) Int. Cl. G01N 33/50

(52) U.S. Cl. 436/68

(57) ABSTRACT

Nitric oxide is measured in blood as a marker of tissue blood flow and oxygenation. Where nitric oxide is measured to be below physiological or to be below average, in a patient having a disease associated with oxygen delivery deficiency, nitric oxide is infused alone or with nitrite. Where nitric oxide is measured to be normal, nitrite is infused. The storage time of whole blood or red blood cells is increased and the vasodilator response of outdated or NO depleted whole blood and red blood cells are increased by treatment with nitric oxide and/or nitrite. A patient in need of a transfusion is transfused with whole blood or red blood cells treated to increase vasodilator response therein.

### CROSS-REFERENCE TO RELATED APPLICATION

This application claims the benefit of U.S. Provisional Application No. 60/383,595, filed May 29, 2002.

### TECHNICAL FIELD

This invention is directed to measurement of blood gases and blood treatments and therapies based thereon.

### BACKGROUND OF THE INVENTION

Whereas O₂ and CO₂ measurements are typically used to assess the severity of human illness and the response to therapy, there is a well known disconnect between pO₂ and tissue oxygenation.

It has not heretofore been conceived that nitric oxide (NO) level in blood provides a measure of tissue oxygenation (determined by blood flow) and measurement NO in blood has not been previously linked to assessment of human condition and to a therapeutic response.

Moreover, it is not heretofore been determined that NO levels in blood bank blood deplete over time and that restoring NO content of red blood cells can increase storage times for whole blood and red blood cells beyond six weeks and increase the vasoactivity of stored blood and red blood cells thereby mitigating transfusion risk.

### SUMMARY OF THE INVENTION

Data has been developed by the inventors herein which shows that NO is a critical component of the human respiratory cycle and is a blood gas which functions to regulate O₂ delivery, i.e., that the NO level adds significantly to the blood gas by providing a measure of tissue oxygenation (determined by blood flow) and that red blood cells and the loading of them with NO contributes significantly to the classical physiological response of hypoxic vasodilation and hyperoxic vasoconstriction. Morever, data has been developed that shows NO binding to hemes and thiols of hemoglobin varies as a function of hemoglobin saturation with oxygen to dilate or constrict pulmonary and systemic arteries in vivo. Thus data has been developed which provides a mechanistic basis for the longstanding mystery of the failure of pO₂ to determine tissue oxygenation. Furthermore, data has been developed by the inventors herein that blood bank red blood cells become depleted in NO over time resulting in impaired vasodilator response.

It has thus been discovered herein that measurement of NO (in conjunction with O₂/CO₂) in blood is a marker of tissue blood flow and oxygenation and that the ability to monitor and manipulate levels of NO in red blood cells is useful in the assessment of blood gases, in the assessment of well being, in the diagnosis and treatment of diseases of the heart, lung and blood associated with oxygen deficiency and in the rational development of therapeutics, including NO donors, erythropoietin therapy and blood substitutes. Furthermore, it has been discovered that the function of red blood cells depends on their NO content; and that by introducing NO or nitrite therein, storage time for red blood cells and whole blood is increased; and that treatment of outdated red blood cells with NO or nitrite normalizes vasodilation response thereto, thereby providing a method for increasing storage times for whole blood and red blood cells and mitigating morbidity and mortality risks of transfusions.

One embodiment of the invention herein, denoted the first embodiment is directed to a method of determining from blood, levels of blood gas components corresponding to condition selected from the group consisting of physiological and pathological conditions of a patient, comprising measuring NO level in conjunction with measuring pO₂ and pCO₂ in blood of the patient as a marker of tissue blood flow and oxygenation

Another embodiment of the invention herein, denoted the second embodiment, is directed to a method of treating a disease associated with oxygen deficiency in heart, lung or blood in a patient having such disease, comprising infusing into the patient a therapeutically effective amount of an anaerobic solution of nitric oxide at a rate in the range of 1 to 500 nmol nitric oxide/min.

Another embodiment of the invention herein, denoted the third embodiment, is directed to a method of treating a disease associated with oxygen deficiency in heart, lung or blood, in a patient having such disease, comprising administering to said patient a therapeutically effective amount of an anaerobic solution of NO at a rate in the range of 1 to 500 nmol nitric oxide/min and nitrite in an amount which facilitates the oxygen delivery improving activity of the NO.

Another embodiment of the invention herein, denoted the fourth embodiment, is directed to a method of treating a disease associated with oxygen deficiency in heart, lung or blood, in a patient having such disease and a physiological (normal) level for that patient of NO in blood, comprising administering a therapeutically effective amount of an anaerobic solution of nite in an amount which facilitates the oxygen delivery activity (blood flow increasing activity) of the NO.

Still another embodiment of the invention herein, denoted the fifth embodiment, is directed to increasing the storage time of whole blood or red blood cells comprising admixing outdated or NO depleted whole blood or red blood cells with a solution of NO or nitrite to replete or increase vasodilator response in the whole blood or red blood cells.

Still another embodiment of the invention herein denoted the sixth embodiment, is directed to a method of transfusing a patient in need of a transfusion comprising treating whole blood or red blood cells depleted in NO with a solution of NO and/or nitrite, to increase or replete vasodilator response therein and transfusing the treated whole blood or red blood cells into the patient.

A disease associated with oxygen deficiency in a patient is used herein to mean a disease where oxygen delivery is deficient as indicated by measurement of an NO level at least 10% below baseline for physiological condition for the patient or by measurement of NO level for the patient which is at least 10% lower than the average for a group, as measured in the first embodiment.

For the fourth embodiment, NO in a normal level for that patient means NO for that patient for physiological condition.

The term “outdated whole blood or red blood cells” is used herein to mean exceeding the storage periods described hereafter or loss of NO by more than 10% compared to NO level in fresh (normal) blood or inability to recoup SNO on a deoxygenation-oxygenation cycle. In explanation of the latter, if red blood cells are deoxygenated for long periods (e.g., by letting set for one hour), NO in the red blood cells cannot form SNO because the position of NO on hemoglobin moves so it is not in facile contact with cysteine. In other words, over time red blood cells lose ability to make SNO.

The term “whole blood or red blood cells depleted in NO” is used herein to mean loss of NO by more than 10% compared to NO level in fresh (normal) blood or inability to recoup SNO on a deoxygenation-oxygenation cycle as described above.

The term “replete or increase vasodilator response in whole blood or red blood cells” is used herein to mean increasing NO level by at least 10%.

The term “storage time” is used herein to mean time from phlebotomy.

### BRIEF DESCRIPTION OF THE DRAWINGS

FIG. 1 compares NO in control with that in outdated samples of red blood cells and shows results of Example V.

FIG. 2 compares vasodilator response for PBS and nitrite treated outdated red blood cells and shows results of Example V.

FIG. 3 compares percent relaxation of control outdated red blood cells and nitrite and NO treated outdated red blood cells and shows results of Example V.

### DETAILED DESCRIPTION

We turn now to the first embodiment of the invention herein, that is the method of determining from blood, levels of blood gas corresponding to condition selected from the group consisting of physiological and pathological conditions of a patient, comprising measuring NO level in conjunction with measuring pO₂ and pCO₂ in blood of the patient as a marker of tissue blood flow and oxygenation.

Measurement of NO level in blood of a patient can be carried out in venous blood of the patient or in arterial (mixed venous) blood of the patient. Human venous blood can be drawn, for example, via an antecubital vein.

Measurement of NO in blood is readily carried out, for example, by photolysis-chemiluminescence, in a DAF-2 assay or by electron paramagnetic resonance spectroscopy (EPR), for example to determine ratio of SNO or NO to hemoglobin.

For assay by photolysis-chemiluminescence, hemolysate protein (about 99% hemoglobin) from red blood cells was partially purified by gel filtration (5000 g, 60 seconds) over a G-25 fine Sephadex chromatography column (25-fold volume excess) equilibrated with phosphate buffered saline (PBS) (pH 7.40, with 0.5 mM EDTA). An airtight Hamilton glass syringe was used to transfer 90 μl of sample (final [hemoglobin], 100 μM) for injection as described in McMahon, T. J. and Stamler, J. S., Methods Enzymol 301, 99-114 (1999).

For DAF-2 assays, one half of the homolysate was adjusted to 100 μM hemoglobin with PBS-containing diaminofluoroscein-2 (DAF-2) (final pH 7.4) either with or without HgCl, and incubated 10 minutes. Centricon 10 kDa filters (10, 600 g, 20 min) were then used to exclude hemoglobin prior to fluorescence measurement. The filtrates were transferred to microplates and treated with acid (0.4N HCl final concentration) to generate nitrosating equivalents and then with excess NaOH to maximize fluorescence (excitation and emission: 485 nm and 520 nm, respectively). Standard curves were constructed using S-nitrosohemoglobin as described in McMahon, T. J., et al. J. Biol. Chem 275, 16738-45 (2000).

For assay by EPR, spectroscopy was carried out as described in Gow, A. J., et al, Proc. Natl. Acad. Scie. USA 96, 9027-9032 (1999). SNO-oxyhemoglobin samples (0.5 mM protein) for comparison were prepared as described in Jia, L., et al, Nature, 380, 221-226 (1996).

Photolysis-based and EPR techniques which are well-established probes for NO/hemoglobin interactions, are preferred.

Testing has indicated a wide variation in physiological (natural) NO level among individuals, usually a SNO/hemoglobin mole ratio ranging from 0.0001 to about 0.0029, more typically ranging from 0.0005 to about 0.002. Because of the inter-individual variability in NO levels, it is important to compare the NO level for an individual to the individual over time as well as to group at large.

Measuring of pO₂ and pCO₂ can be carried out conventionally.

We turn now to the second, third and fourth embodiments herein.

Each of the second, third and fourth embodiments, is directed to treating a disease associated with oxygen deficiency in heart, lung or blood in a patient having such disease. As indicated above, a disease associated with oxygen deficiency in a patient is a disease where oxygen delivery is deficient as indicated by measurement of level of NO at least 10% below that for physiological (non-pathological) condition (for that individual, or if such data is not available, below the average for a group at large, e.g., lower than 1 mol NO per 1000 moles hemoglobin) as measured in the first embodiment.

Diseases associated with oxygen deficiency in heart include angina.

Diseases associated with oxygen deficiency in lung include pulmonary hypertension.

Diseases associated with oxygen deficiency in blood include sickle cell disease.

We turn now the second embodiment herein which is directed at a method of treating a disease associated with oxygen deficiency in heart, lung or blood, in a patient having such disease, comprising infusing into the patient a therapeutically effective amount of an anaerobic solution of nitric oxide at a rate in the range of 1 to 500 nmol NO/min.

The anaerobic solution of nitric oxide can, e.g., be a saturated solution of NO (1.5 mM NO) in saline (0.9% NaCl) and can be made anaerobic by bubbling inert gas through the solvent before introduction of NO therein.

The therapeutically effective amount is a blood flow increasing (oxygen delivery to tissue improving) amount at a rate ranging from 1 to 500 nmol NO/min, preferably from 1 to 10 nmol NO/min, for as long as improvement or benefit occurs. The amount is an NO repleting amount for red blood cells. The concentration of NO should be one that has no acute effect on systemic blood pressure or systemic hemodynamics. Previously, a solution of NO has been infused at a rate of 0.75 to 36 micromoles/min, with the higher concentration being viewed as effective; this range can have an acute effect on systemic blood pressure or systemic hemodynamics.

We turn now to the third embodiment herein which is directed at a method of treating a disease associated with oxygen deficiency in heart, lung or blood, in a patient having such disease, comprising administering to said patient, e.g., by infusing into the patient, a therapeutically effective amount of an anaerobic solution of NO at a rate in the range of 1 to 500 nmol NO/min, preferably from 1 to 10 nmol NO/min, and nitrite in an amount which facilitates the oxygen delivery improving activity of NO (as manifested by increased blood flow).

The administration of the NO and therapeutically effective amount thereof is the same as for the second embodiment.

The nitrite is any that is soluble in and compatible with blood and can be, for example, inorganic nitrite such as sodium nitrite or potassium nitrite or calcium nitrite and is preferably present in the anaerobic solution of NO, e.g, in saline, which is infused and is present in an amount which improves the oxygen delivery activity identified with the NO infusion by facilitating the formation of S-nitrosohemoglobin. The benefit of nitrite is indicated by data developed by the inventors which shows that in the presence of nitrite, the biological activity identified with NO is facilitated. The nitrite is administered at a rate of 1 nmol to 10 μM nitrite/minute which improves the oxygen delivery improving effect of the NO, e.g., in an amount of 20 to 150 fold the NO concentration.

We turn now to the fourth embodiment herein which is directed to a method of treating a disease associated with oxygen deficiency in heart, lung or blood in a patient having that disease and a physiological level for that patient of NO in blood, comprising administering to the patient a therapeutically effective amount of an anaerobic solution of nitrite in an amount which facilitates the oxygen delivery activity (blood flow increasing activity) of the NO (by facilitating the formation of S-nitrosohemoglobin).

The nitrite is preferably administered by infusing an anaerobic solution thereof into the patient, e.g., in saline made anaerobic by bubbling inert gas therethrough before admixture of nitrite.

The nitrite is any that is soluble in and compatible with blood and can be, for example, inorganic nitrite such as sodium nitrite or potassium nitrite or calcium nitrite and is infused in a therapeutically effective amount which improves the oxygen delivery improving activity of NO by facilitating formation of S-nitrosohemoglobin, e.g., at a rate in the range of 1 nmol to 10 μM nitrite/minute.

For the second and third embodiments, the NO is preferably administered from a stock solution of 1.5 mM NO (saturated solution) at a concentration of 1.5 mM or lower in saline (0.9% NaCl). Nitrite can be given also from a stock solution as needed to achieve nanomolar to micromolar concentrations.

We turn now to the fifth embodiment of the invention herein, that is a method for increasing the storage time of whole blood or red blood cells comprising admixing outdated or NO depleted whole blood or red blood cells with a solution of NO and/or nitrite, to replete or increase vasodilator response in the whole blood or red blood cells.

Presently, whole blood or red blood cells preserved with citrate-phosphate-dextrose-adenine may be stored for 35 days. Red blood cells preserved with adenine-saline preservative may be stored for 42 days. After storage for these periods, the whole blood and red blood cells are considered outdated and may not be used for transfusion purposes because of concerns of increased morbidity and mortality risks. The present method can be used on whole blood or red blood cells that are outdated or depleted in NO until such time as NO bioactivity is no longer increased, e.g., when the red cells are no longer intact as may be determined under a microscope or by measuring free hemoglobin in a hemolysate. In other words, consecutive treatments are useful on intact red blood cells per se or in whole blood so long as NO bioactivity including vasodilator function is increased. The NO bioactivity can include activity from SNO, NO, NOₓ, NO⁺ and NO⁻.

The NO and/or nitrite is admixed by admixing a solution of NO or nitrite in saline or phosphate buffered saline e.g., as 1 mM to 1.5 mM NO and/or nitrite, to load the blood product with NO or nitrite to a molar ratio of NO and/or nitrite to hemoglobin ranging from 1:10 to 1:1,000. Suitable nitrites are those discussed in conjunction with the fourth embodiment herein. Preferably the solution of NO or nitrite is anaerobic as this makes the administration more efficient. Anaerobicity can be effected by admixing anaerobic solvent with the NO or nitrite under anaerobic conditions. The solvent can be made anaerobic by bubbling inert gas, e.g., argon, through the solvent before introduction of NO or nitrite therein. Whole blood and red blood cells which have been deoxygenated by exposure to the atmosphere and vortexing (mixing), or can be used in deoxygenated state whereupon oxygenation occurs in the body or in vitro.

The NO and/or nitrite treatment is carried out to restore or increase vasodilator response as may be measured by blood flow increase or clinical outcome.

We turn now to the sixth embodiment of the invention herein, that is a method of transfusing a patient in need of a transfusion comprising treating whole blood or red blood cells depleted in NO with a solution of NO and/or nitrite, to increase vasodilator response and transfusing the treated whole blood or red blood cells into the patient.

A patient is in need of a transfusion is a patient who has lost or is losing blood or one in need of removal of waste products of the body in case of failure of renal functioning or needs removal of toxic substance from blood in the case of poisoning or is in need of red blood cells or hemoglobin to treat any disease associated with impairment of nitric oxide or oxygen, e.g., angina or stroke.

The term “whole blood or red blood depleted in NO” is used herein to mean at least 10% less relaxation in the test described in McMahon, T. J., et alt Nature Medicine 8, 711-717 (2002) compared to when the whole blood or red blood cells are first donated.

The solution of NO and/or nitrite can be formed by admixing NO or nitrite with saline or phosphate buffered saline, e.g., as 1 nM to 1.5 mM NO and/or nitrite, to load the blood product with NO and/or nitrite to a ratio of NO and/or nitrite to hemoglobin ranging from 1:10 to 1:1,000. Suitable nitrites are those discussed in conjunction with the fourth embodiment herein. Preferably the solution of NO and/or nitrite is made anaerobic as discussed in the description of the fifth embodiment, and admixing of anaerobic solution of NO and/or nitrite with blood product is carried out under anaerobic conditions.

Whole blood and red blood cells which have been deoxygenated can be oxygenated by exposure to air (the atmosphere) and vortexing (musing) or can be used in deoxygenated state whereupon oxygenation occurs in the body.

The transfusing can be carried out by conventional means.

Background of the invention herein and elements of the invention herein are set forth in McMahon, T. J., et at, Nature Medicine 8, 711-717 (2002) which is incorporated herein by reference.

The whole of U.S. Provisional Application No. 60/383,595 including Appendix A thereto is incorporated herein by reference.

The invention is illustrated by the following working examples.

### EXAMPLE I

Measurements of NO were made in samples of blood of subjects of normal health, i.e., no pathological condition. Measurement was carried out by DAF-2 and photolysis chemilminescene as described above. The results show inter-individual variability in NO levels and thus the importance of comparing NO level to both the group at large and the individual over time.

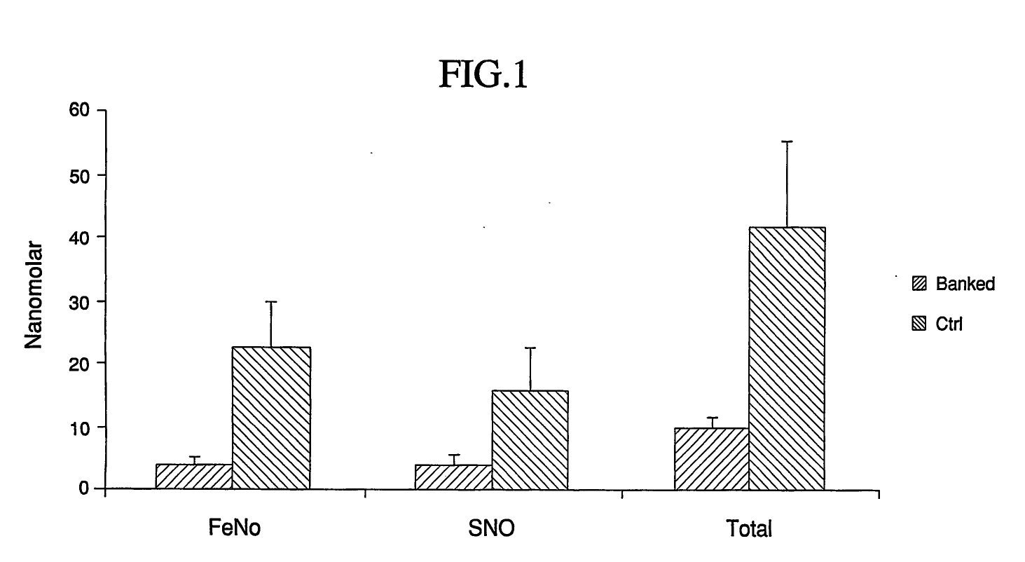

EPR assay was carried out with Fe(II) NO spiked hemoglobin samples prepared in PBS pH 7.4, 300 μM nitrite, with a heme concentration of 1 mM, and an Fe(II) NO concentration of 50 μL. Comparison was to SNO-oxyhemoglobin samples (0.5 mM protein) prepared as described in Jia, L., et al, Nature 380, 221-226 (1996). Results as shown in FIGS. 1c and 1d of McMahon, T. J., et al, Nature Medicine, 711-717 (2002), indicate that in presence of nitrite, oxygen delivery improving activity identified with NO, is facilitated.

### EXAMPLE II

A 65-year old male is admitted to a hospital with unstable angina. The patient is given I.V. nitroglycerin, heparin and a beta blocker. However, the patient continues to experience chest pain at rest. The patient's normal NO level is known from past testing. Measurement of the patient's NO level shows it is below normal Alternatively, the patient's normal NO level is not known from past testing but measurement of the patient's NO level shows it is lower than average. Infusion at a rate of 5 nmol/min of NO in 0.9% NaCl is effected. The chest pain resolves.

When nitrite is additionally given at a rate of 100 nmol/min, the chest pain resolves more quickly.

### EXAMPLE III

A 27-year old female with primary pulmonary hypertension class II presents complaining with shortness of breath. The patient's normal NO level is know from prior testing. Measurement of the patient's NO level shows it is below normal. Infusion of NO in 0.9% NaCl at a rate of 5 nmol/min of NO is effected. The shortness of breath symptom resolves. After three days of therapy, pulmonary artery pressure has dropped 5 mm of mercury.

### EXAMPLE IV

A patient presents with sickle cell disease presents with hypoxemia. NO level is measured and found to be the same as in previous testing. An anaerobic saturated solution of sodium nitrite in saline in infused at a rate of 100 nmol/min. Improved oxygen delivery occurs.

### EXAMPLE V

Nine samples of red blood cells were obtained from the blood bank after 6 weeks storage at which time the samples were viewed as outdated. Levels of NO in the red blood cells as measured by photolysis chemiluminescence are depleted very significantly as compared to control (freshly donated samples). The results are shown in FIG. 1.

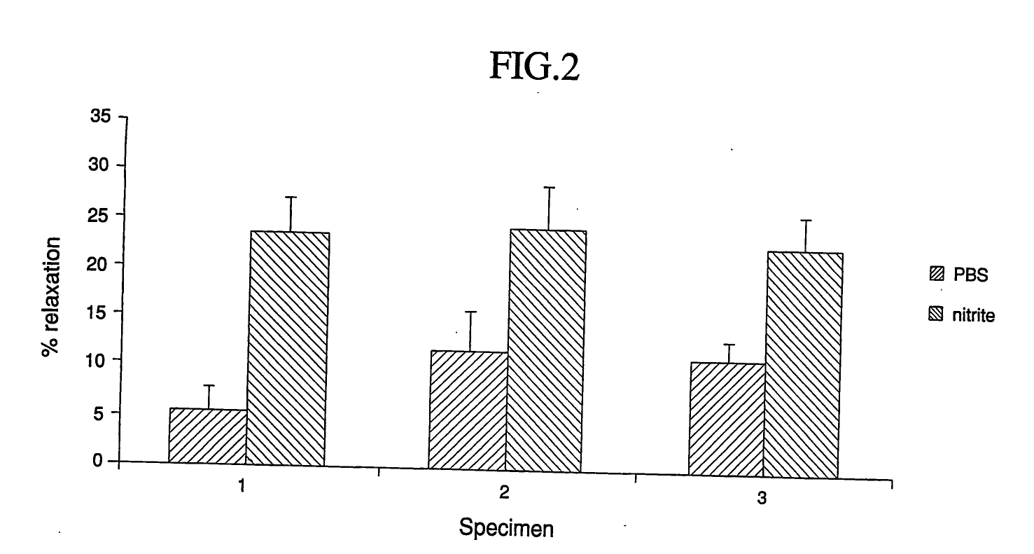

These samples of outdated red blood cells with depleted NO levels showed impaired vasodilator responses in assays carried out by adding red blood cells to standard organ chamber bioassays at low pO₂ as described in McMahon, T. J., et al, Nature Medicine 8, 711-717 (2002). In these assays, normal vasodilator responses were about 25% relaxation. The samples were determined to provide 5% and 10% relaxation. The samples were deoxygenated by placing them in a bioassay bath at low pO₂. Then, in paired samples, sodium nitrite (1:200 molar ratio of nitite to hemoglobin in phosphate buffered saline) was added to the deoxygenated red blood cells for 10 minutes. The samples were then re-oxygenated by reintroducing air followed by shaking. This treatment was found to normalize vasodilator response whereas treatment with phosphate buffered saline (PBS) without NO or nitrite had no effect on vasodilator response. The results are shown in FIG. 2. Nitrite was found to have no effect on vasodilator response of native fresh red blood cells.

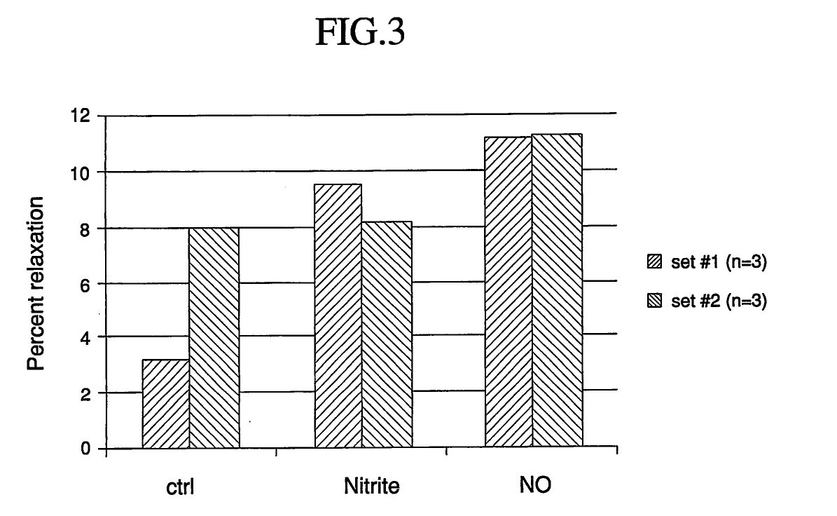

Two sets of 3 samples each of outdated red blood cells showed impaired relaxation in testing as described in McMahon, T. J., et al, Nature Medicine 8, 711-717 (2002) (3% and 8% relaxation compared to about 15% for fresh red blood cells). Samples from each set were deoxygenated by bubbling argon gas therethrough and then admixed with PBS, PBS with NO dissolved therein or PBS with sodium nitrite dissolved therein. The treatments loaded the red blood cells with 1:250 molar ratio NO or nitrite to hemoglobin. The red blood cells were then oxygenated by exposure to air. The NO and nitrite repleted red blood cells showed improved and effectively normalized relaxations. Results are shown in FIG. 3.

### EXAMPLE VI

Red cells are incubated at a molar ratio of 1:250 NO to hemoglobin for 10 minutes at weekly intervals. At 8 weeks, NO levels and vasodilator response are preserved.

### EXAMPLE VII

A 59-year old with severe coronary artery disease receives a transfusion with blood 5 weeks old. Blood pressure rises by 5 mm Hg. The patient experiences chest pain. A second unit doped with NO (1:250 molar ratio of NO to hemoglobin) is given and chest pain is not experienced.

Variations

Many variations of the above wilt be obvious to those skilled in the art. Thus, the invention is defined by the claims.

What is claimed is:

1. A method of determining from blood, levels of blood gas component corresponding to condition selected from the group consisting of physiological and pathological conditions of a patient, comprising measuring NO level in blood of the patient as a marker of tissue blood flow and oxygenation.

2. A method of treating a disease associated with oxygen deficiency in heart, lung or blood in a patient having such disease, comprising infusing into the patient a therapeutically effective amount of an anaerobic solution of nitric oxide at a rate in the range of 1 to 500 nmol nitric oxide/min.

3. A method of treating a disease associated with oxygen deficiency in heart, lung or blood in a patient having such a disease, comprising administering to said patient a therapeutically effective amount of an anaerobic solution of nitric oxide at a rate in the range of 1 to 500 nmol nitric oxide/min, and nitrite, the nitrite being present in an amount which facilitates the oxygen delivery improving activity of the nitric oxide.

4. A method of treating a disease associated with oxygen deficiency in heart, lung or blood, in a patient having such disease and a physiological level in blood for that patient of nitric oxide, comprising administering a therapeutically effective amount of an anaerobic solution of nitrite to facilitate the oxygen delivery activity of the nitric oxide.

5. A method for increasing the storage time of whole blood or red blood cells comprising admixing outdated or NO depleted whole blood or red blood cells with a solution of NO and/or nitrite, to replete or increase vasodilator response in the whole blood or red blood cells.

6. A method of transfusing a patient in need of a transfusion, comprising treating whole blood or red blood cells depleted in NO and oxygen with a solution of NO and/or nitrite, to increase vasodilator response therein, and transfusing the treated whole blood or red blood cells into the patient.
